(19) United States
(12) Patent Application Publication
(10) Pub. No.: US 2018/0268943 A1
(43) Pub. Date: Sep. 20, 2018

(54) Black Box Warning Notification System and Method

(71) Applicants: Kimberly Grant, Orlando, FL (US); Syed Mobin, Windermere, FL (US)

(72) Inventors: Kimberly Grant, Orlando, FL (US); Syed Mobin, Windermere, FL (US)

(21) Appl. No.: 15/926,445

(22) Filed: Mar. 20, 2018

Related U.S. Application Data
(60) Provisional application No. 62/473,625, filed on Mar. 20, 2017.

(51) Int. Cl. G16H 70/40; G16H 10/65

(52) U.S. Cl. CPC G16H 70/40; G16H 10/65

(57) ABSTRACT

A black box warning notification system and method. The system includes a processor, a database comprising a non-transitory computer readable medium, and a logic stored in the non-transitory computer readable medium that, when executed by the processor, causes the system to perform a method. The method includes receiving a first input from a user device comprising one or more medications, receiving a second input from the user device comprising one or more medical conditions, searching a list of stored medication labels for a matching medication that matches the first input, searching a stored label of the matching medication for the one or more medical conditions, and, if a word of the medical condition matches a word on the stored label of the matching medication, then wirelessly transmitting a warning notification to the user device. In one embodiment, the user device displays a specific warning notification corresponding to the exactness of the match between the medical condition and warning label text.

### CROSS REFERENCE TO RELATED APPLICATIONS

This application claims the benefit of U.S. Provisional Application No. 62/473,625 filed on Mar. 20, 2017. The above identified patent application is herein incorporated by reference in its entirety to provide continuity of disclosure.

### BACKGROUND OF THE INVENTION

The present invention relates to a system and method for providing medication-related information. More specifically, the present invention provides a system and method for determining whether a particular medication includes risks of serious side-effects.

Many prescription drugs can cause mild to moderate side effects that may adversely affect the individual consuming the drug. Some prescription drugs can cause side effects in any individual, and such side effects are often mild enough to deal with, or rare enough that they only occur in a small number of patients. Other prescription drugs can cause side effects that only manifest in individuals that have a corresponding medical condition that causes that particular side effect to arise. For example, drugs which include anti-platelet agents help to reduce the risk of cardiovascular death or stroke in patients with acute coronary syndrome by preventing the formation of potentially life-threatening blood clots. However, such anti-platelet agents can cause significant bleeding due to their anti-clotting properties, and such bleeding can be fatal to individuals with active pathological bleeding or a history of cranial hemorrhaging.

In the foregoing scenario, and with all prescription drugs, it is important for the individual consuming the prescription drug to be notified of any potential serious side effects. Some side effects are listed on the medication packaging at the discretion of the drug manufacture for individuals to read at their own preference. However, the Food and Drug Administration (FDA) has determined that serious or life-threatening side effects are required to be prominently displayed on the drug packaging. To that end, the FDA requires a boxed warning, often referred to as a “black box warning” due to the black border that surrounds the text of the warning, to be placed on the packaging of a prescription drug if there are medical studies that indicate the drug carries a significant risk of serious or life-threatening side effects. The black box warning includes warning text surrounded by a box or border so that it is prominently displayed and easy to identify.

Despite the importance of these black box warnings, some individuals may be uninformed about the boxed warning on the medication they consume. One purpose of the boxed warning is to alert doctors or other drug prescribers of potential serious risks regarding the drug so that the risks can be considered prior to prescribing the drug. However, the prescribing doctor does not always have the patient's entire medical history at hand, and may be unaware of a particular serious complication that may occur. Additionally, boxed warnings are often added to existing prescription drugs after an individual has already been prescribed the drug. For example, the FDA often requires a boxed warning to be added to an existing prescription drug if new medical studies are conducted that determine there is a serious or life-threatening risk associated with the drug. Users of the prescription drug are not necessarily notified when a new boxed warning is added, and as such may continue consuming the drug despite updated information regarding serious risks and potentially life-threatening side effects. In view of the above concerns, it is necessary to provide a system and method for notifying individuals of black box warnings on the medications they consume, and to further notify the individual if the individual has an existing medical condition that corresponds to a particular black box warning.

### SUMMARY OF THE INVENTION

In view of the foregoing disadvantages inherent in the known types of medication notification systems now present in the prior art, the present invention provides a black box warning notification system and method wherein the same can be utilized for providing convenience for the user when determining whether their medication or potential medication can cause serious side effects. The present system includes a black box warning notification system comprising a processor, a database comprising a non-transitory computer readable medium operatively connected to the processor, and a logic stored in the non-transitory computer readable medium that, when executed by the processor, causes the system to perform a method. The method includes receiving a first input from a user device comprising one or more medications, receiving a second input from the user device comprising one or more medical conditions, searching a list of stored medication labels for a matching medication that matches the first input, searching a stored label of the matching medication for the one or more medical conditions, and, if a word of the medical condition matches a word on the stored label of the matching medication, then wirelessly transmitting a warning notification to the user device.

An object of the present invention is to provide a black box warning notification system that is accessible by patients as well as approved medication prescribers.

Another object of the present invention is to provide a black box warning notification system that allows users to search for and read the full label for a particular prescription drug.

A further object of the present invention is to provide a black box warning notification system that searches a database containing a comprehensive list of medications and their warning labels, as well as a comprehensive list of medical conditions.

Still a further object of the present invention is to provide a black box warning notification system that displays different levels of warnings depending upon the match found in the warning label compared to a particular medical condition.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a block diagram of the black box warning notification system and method.

FIG. 2 shows a flowchart of the method steps of the black box warning notification system and method.

FIG. 3 shows a top-down view of an example user interface of the black box warning notification system and method.

FIG. 4 shows an alternate top-down view of an example user interface of the black box warning notification system and method.

FIG. 5 shows another alternate top-down view of an example user interface of the black box warning notification system and method.

### DETAILED DESCRIPTION OF THE INVENTION

Reference is made herein to the attached drawings. Like reference numerals are used throughout the drawings to depict like or similar elements of the black box warning notification system and method. For the purposes of presenting a brief and clear description of the present invention, the preferred embodiment will be discussed as used for determining possible side effects for particular medications. The figures are intended for representative purposes only and should not be considered to be limiting in any respect.

According to some embodiments, the operations, techniques, and/or components described herein can be implemented by an electronic device, which can include one or more special-purpose computing devices. The special-purpose computing devices can be hard-wired to perform the operations, techniques, and/or components described herein, or can include digital electronic devices such as one or more application-specific integrated circuits (ASICs) or field programmable gate arrays (FPGAs) that are persistently programmed to perform the operations, techniques and/or components described herein, or can include one or more general purpose hardware processors programmed to perform such features of the present disclosure pursuant to program instructions in firmware, memory, other storage, or a combination. Such special-purpose computing devices can also combine custom hard-wired logic, ASICs, or FPGAs with custom programming to accomplish the technique and other features of the present disclosure. The special-purpose computing devices can be desktop computer systems, portable computer systems, handheld devices, networking devices, or any other device that incorporates hard-wired and/or program logic to implement the techniques and other features of the present disclosure.

In the interests of economy, the present disclosure refers to “a computer-readable medium,” “a processor,” and so on. However, this should not be read as limiting in any way as the present disclosure contemplates embodiments of the present invention utilizing “one or more computer-readable media,” “one or more processors,” and so on. Unless specifically limited to a single unit, “a” is intended to be equivalent to “one or more” throughout the present disclosure.

As used herein, “patient” refers to an individual consuming a prescription drug that has been prescribed to them by a licensed prescription drug prescriber.

As used herein, “prescriber” refers to an approved prescribing physician or other individual licensed to prescribe prescription drugs.

As used herein, “black box warning” refers to the boxed warning label required by the FDA when there is reasonable evidence of an association of a serious hazard with the drug.

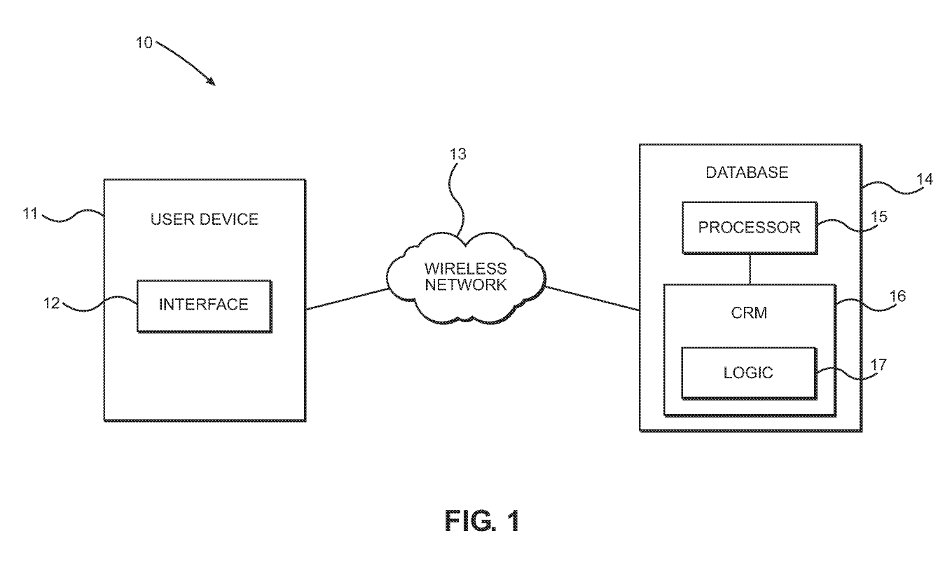

Referring now to FIG. 1, there is shown a block diagram of the black box warning notification system and method. The system 10 is accessible via a user device 11 including an interface 12. The interface 12 includes at least a display screen and an input mechanism such as a touchscreen, keyboard, or the like. The system 10 includes a database 14 having a processor 15 operatively connected to a computer readable medium 16. A logic 17 is stored on the computer readable medium 16. The processor 15 is configured to execute the instructions provided by the logic 17 in order to perform a method according to the present invention. The database 14 is further configured to communicate with the user device 11 via a wireless network, such as a WiFi or other internet connection. The database 14 also includes a comprehensive list of medications and medical conditions that can be updated as needed.

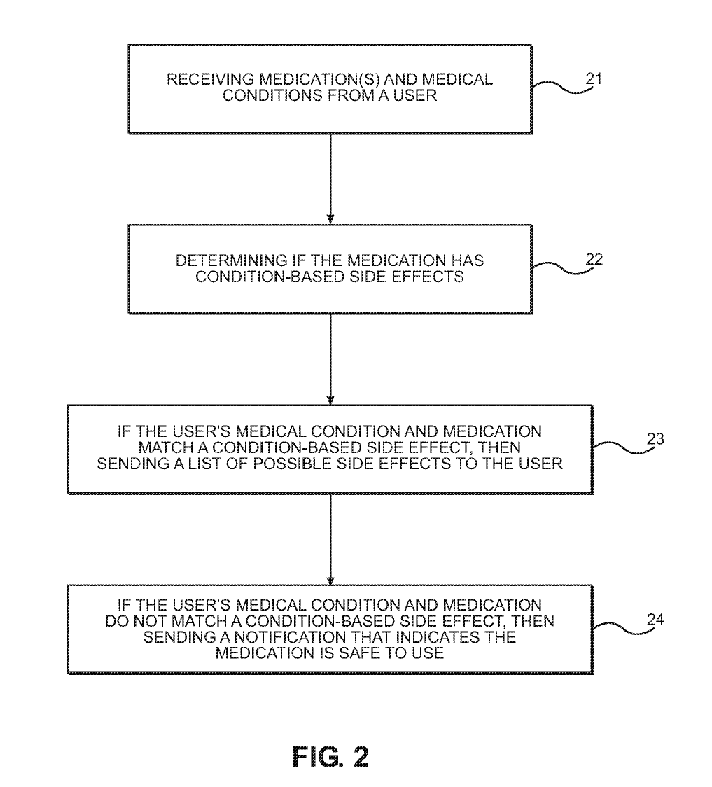

Referring now to FIG. 2, there is shown a flowchart of the method steps of the black box warning notification system and method. As an initial step, the system receives 21 medications and medical conditions from a user device. The medications or medical conditions can be entered directly into the user device and transmitted to the system. In an alternate embodiment, the medications or medical conditions can be selected from a preloaded list. The system then determines 22 whether the medication has a condition-based side effect. The system accomplishes this by searching the database for a match on the label of the medication to the name of the medical condition. If a match is found between the medical condition and the medication's warning label, a warning notification is sent 23 to the user. The warning notification can be overlaid on an image of the medication's label, so that users may read the entirety of the label. In some embodiments, if no match is found between the medical condition and the medication warning label, then a safe-to-use notification is sent 24 to the user, indicating that there are no serious known side effects.

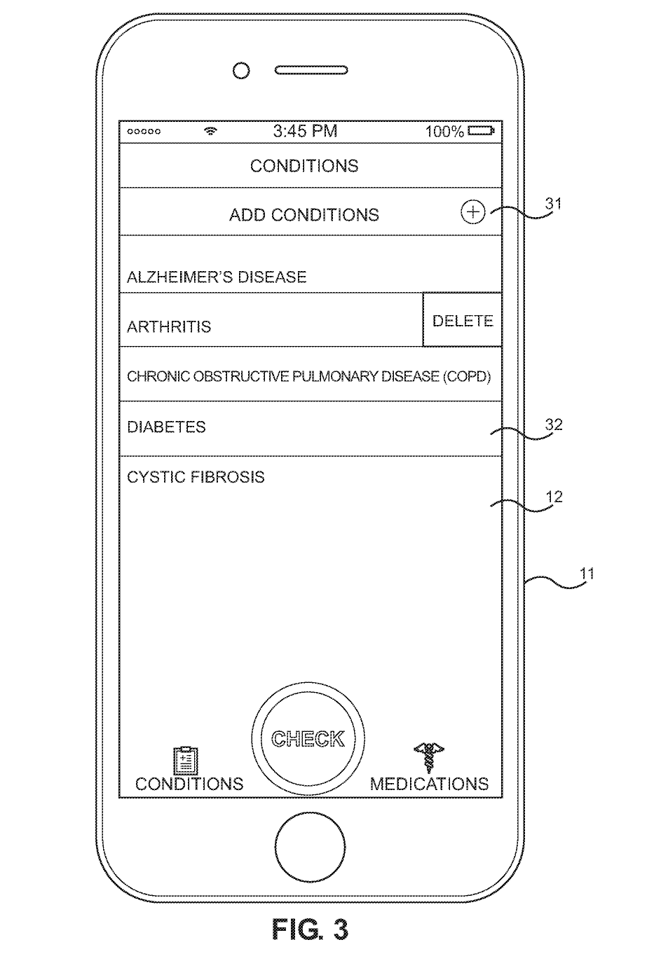

Referring now to FIG. 3, there is shown a top-down view of an example user interface of the black box warning notification system and method. The user device 11 includes an interface 12 which provides for the input and display of medical conditions. The system may provide an input selection 31 that can be selected to add medical conditions. For example, an individual may pick from a list of medical conditions, or manually enter a medical condition via the interface 12. Once inputted by the user, the interface 12 displays a list 32 of medical conditions that can easily be reviewed or edited by the user.

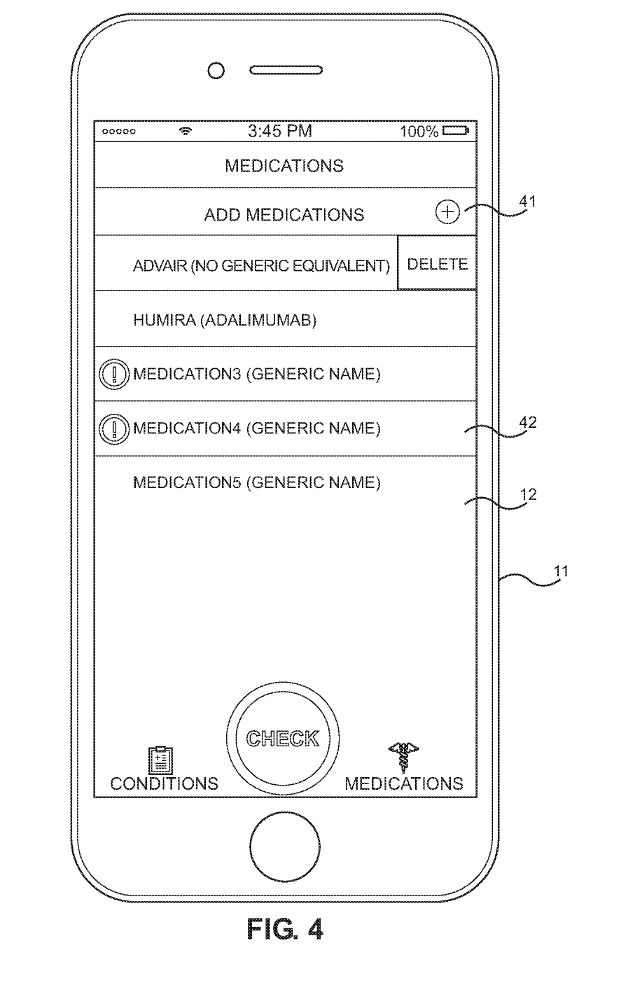

Referring now to FIG. 4, there is shown an alternate top-down view of an example user interface of the black box warning notification system and method. The interface 12 of the user device 11 is further utilized by the user to input medication names. The system is configured to recognize both brand names and generic names for particular medications. Similar to the list of medical conditions, the medications can be manually inputted or chosen from a list, and the user device 11 displays a list 42 of inputted or chosen medications that can be reviewed and edited at any time.

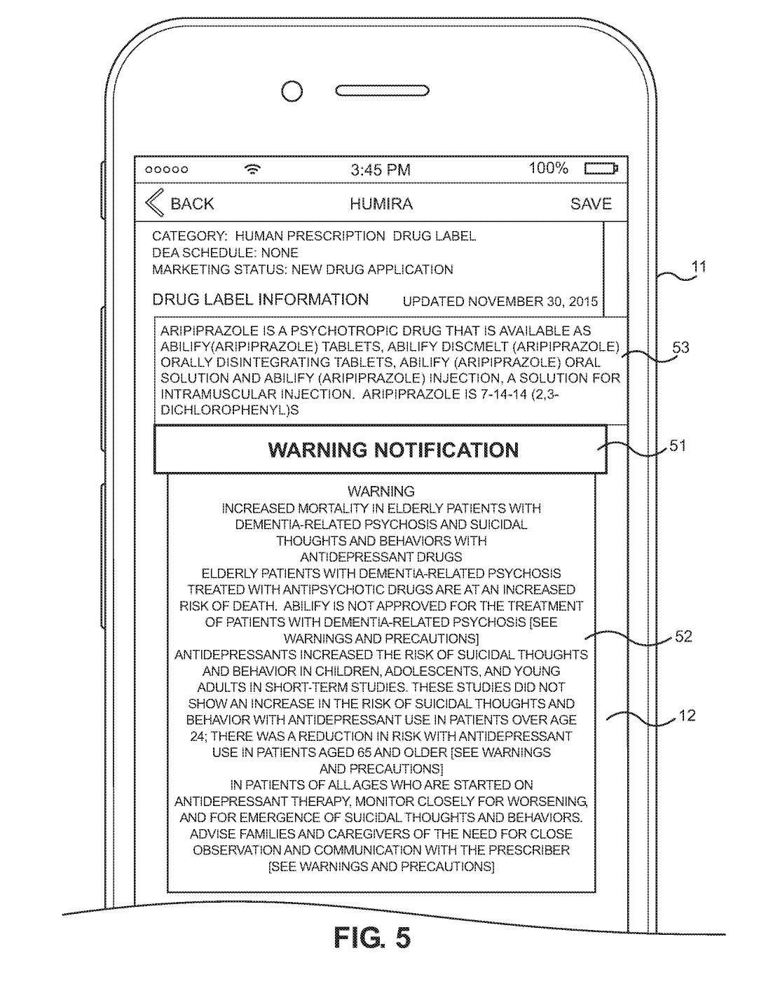

Referring now to FIG. 5, there is shown another alternate top-down view of an example user interface of the black box warning notification system and method. The user interface 12 is configured to display the label 53 and the black box warning 52 of a particular medication when accessed by the user. It is contemplated that present invention may be utilized by both patients and prescribers. Patients can use the system to learn about warning labels on medications they are currently prescribed or may wish to be prescribed in the future, and prescribers can use the system to determine whether there is a serious risk or side effect associated with a particular medication before they prescribe that medication to a patient.

In operation, the logic is configured to search for medical condition matches within the medication label. All portions of the medication label are searched, including the black box warning section 52 and any general warnings and precautions sections, such as a contraindications section. In addition to displaying the medication label, the system is configured to wirelessly transmit and display a notification if a match is found. The type of notification may vary according to the exactness of the match. For example, the warning notification 51 can include a first color, such as yellow, if there is a partial match between the medical condition and the warning label text. The warning notification 51 can include a second color, such as red, if there is an exact match between the medical condition and the warning label text. The system can also highlight the matched words in the warning label, so that the user can easily identify important parts of the warning label that pertain to their medical condition.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A black box warning notification system comprising:
a processor;
a database comprising a non-transitory computer readable medium operatively connected to the processor;
a logic stored in the non-transitory computer readable medium that, when executed by the processor, causes the system to perform a method, the method comprising:
receiving a first input from a user device comprising one or more medications;
receiving a second input from the user device comprising one or more medical conditions;
searching a list of stored medication labels for a matching medication that matches the first input;
searching a stored label of the matching medication for the one or more medical conditions;
if a word of the medical condition matches a word on the stored label of the matching medication, then wirelessly transmitting a warning notification to the user device.

2) The system of claim 1, wherein the method further comprises:
displaying an entire label of the matching medication on the user device.

3) The system of claim 1, wherein the method further comprises:
if less than every word of the medical condition is found in a label of the matched medication, between the medical condition and the warning label is found, then displaying a first warning notification comprising a first color.

4) The system of claim 1, wherein the method further comprises:
If every word of the medical condition is found in a label of the matched medication, then displaying a second warning notification comprising a second color.

5) A non-transitory computer readable medium that, when executed by a processor of a system, causes the system to perform a method, the method comprising:
receiving a first input from a user device comprising one or more medications;
receiving a second input from the user device comprising one or more medical conditions;
searching a list of stored medication labels for a matching medication that matches the first input;
searching a stored label of the matching medication for the one or more medical conditions;
if a word of the medical condition matches a word on the stored label of the matching medication, then wirelessly transmitting a warning notification to the user device.

6) The non-transitory computer readable medium of claim 5, wherein the method further comprises:
displaying an entire label of the matching medication on the user device.

7) The non-transitory computer readable medium of claim 5, wherein the method further comprises:
if less than every word of the medical condition is found in a label of the matched medication, between the medical condition and the warning label is found, then displaying a first warning notification comprising a first color.

8) The non-transitory computer readable medium of claim 5, wherein the method further comprises:
If every word of the medical condition is found in a label of the matched medication, then displaying a second warning notification comprising a second color.

9) A computer-implemented method comprising:
receiving a first input from a user device comprising one or more medications;
receiving a second input from the user device comprising one or more medical conditions;
searching a list of stored medication labels for a matching medication that matches the first input;
searching a stored label of the matching medication for the one or more medical conditions;
if a word of the medical condition matches a word on the stored label of the matching medication, then wirelessly transmitting a warning notification to the user device.

10) The method of claim 9, further comprising:
displaying an entire label of the matching medication on the user device.

11) The method of claim 9, further comprising:
if less than every word of the medical condition is found in a label of the matched medication, between the medical condition and the warning label is found, then displaying a first warning notification comprising a first color.

12) The method of claim 9, further comprising:
if every word of the medical condition is found in a label of the matched medication, then displaying a second warning notification comprising a second color.
